(19) United States
(12) Patent Application Publication
(10) Pub. No.: US 2006/0286171 A1
(43) Pub. Date: Dec. 21, 2006

(54) Bone morphogenetic protein formulations

(76) Inventors: Tianhong Zhou, Basking Ridge, NJ (US); Chunlin Yang, Belle Mead, NJ (US); Jeffery Charles Geesin, Doylestown, PA (US); Aruna Nathan, Bridgewater, NJ (US); Joseph J. Hammer, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/156,153

(22) Filed: Jun. 17, 2005

(51) Int. Cl. A61K 38/17; A61K 31/724; A61K 31/4172; A61K 31/198

(52) U.S. Cl. 424/486; 514/12; 514/58; 514/400; 514/562

(57) ABSTRACT

Protein formulations that can be lyophilized and are stable in organic solvents. The formulations contain bone morphogenetic proteins, lyoprotectants, and oxidation/reduction stabilizers. Optionally, the formulations may also contain solvent environment stabilizers. The protein formulations can be incorporated into a polymeric matrix to make medical devices for delivering the protein, and coatings for medical devices.

### FIELD OF THE INVENTION

The present invention relates to protein formulations that can be lyophilized and are stable in organic solvents. The invention also pertains to protein/polymer drug delivery devices for implantation and controlled local delivery of the protein.

### BACKGROUND OF THE INVENTION

Bone morphogenetic proteins (BMPs) are unique because they induce the differentiation of mesenchymal cells toward cells of the osteoblastic lineage and also enhance the differentiated function of the osteoblast. They have a variety of uses in orthopedic applications.

Bone morphogenic proteins (in both monomeric and dimeric forms) include TGF-β superfamily factors, BMP-2, BMP-4, BMP-6, BMP-7, BMP-12, BMP-14, and recombinant human growth differential factor 5 (rhGDF-5). Also known as morphogenetic protein 52 (MP52), rhGDF-5 for medical applications has been produced using recombinant DNA techniques. The cDNA of rhGDF-5 was first isolated as the TGF-P superfamily (Biochem. Biophy. Res. Comm., Vol. 204, No. 2. 1994). Then, an advanced genetic engineering technology made it possible to prepare MP52 without impairing its bone morphogenetic activity.

Typically, continuous BMP exposure over days and weeks is required to achieve the goals of bone growth induction. So, the BMPs must be formulated with a second compound, typically a polymer, to form a drug delivery matrix. Proteins are larger and more complex than traditional organic and inorganic drugs, possessing multiple functional groups in addition to complex three-dimensional structures. As such, the formulation of such proteins into suitable dosage forms for therapeutic needs poses special problems. For a protein such as rhGDF-5 to remain biologically active, a formulation must preserve the conformational integrity of at least a core sequence of the protein's amino acids while at the same time protecting the protein's multiple functional groups from degradation.

The polymer matrix in a protein/polymer drug delivery formulation not only provides a mechanism to control the protein dosing rate, but also, more importantly, protects unreleased protein from direct contact with bodily fluid which can degrade the protein.

The maintenance of biological activity after the processing steps required in creating a protein/polymer drug delivery formulation is challenging. High temperatures typically used to compound thermoplastic polymers will often denature the protein. So, protein/polymer drug delivery formulations are often made by mixing the protein into a polymer/solvent solution, where the solvent is a solvent for the polymer. The mixing step is then followed by a step to remove the solvent, resulting in a protein/polymer formulation.

Often, however, the exposure of the protein to organic solvent can degrade the protein. Degradation pathways for proteins can involve chemical instability (i.e. any process which involves modification of the protein by bond formation or cleavage resulting in a new chemical entity) or physical instability (i.e. changes in the higher order structure of the protein). Chemical instability can result from deamidation, racemization, hydrolysis, oxidation, beta elimination or disulfide exchange. Physical instability can result from denaturation, aggregation and precipitation, or adsorption, for example. The three most common protein degradation pathways are protein aggregation, deamidation and oxidation. So, a solvent environment stabilizer is crucial to keep the protein stable when exposed to an organic solvent.

In the absence of a solvent environment stabilizer, rhGDF-5 degrades significantly (both chemically and physically) after the exposure to organic solvent such as dioxane and methylene chloride. For example, when the organic solvent is dioxane, this degradation is due to aggregation or precipitation and oxidation at unspecified sites as observed by RP-HPLC and sodium dodecyl sulfate-polyacrylamide gel electrophoresis (SDS-PAGE) under non-reducing condition. Samples of rhGDF-5 that have been exposed to dioxane have similar RP-HPLC chromatogram peak profiles to rhGDF-5 samples that have been incubated with 0.1 percent peroxide solutions. When these samples are examined by SDS-PAGE under non-reducing conditions, only a single band corresponding to the intact rhGDF5 was observed, indicating physical aggregation is the cause of precipitation.

Proteins generally have better stability for long-term storage when they are in a dry state. Freeze-drying, or lyophilization, is a commonly employed technique for preserving proteins that serves to remove water from the protein preparation of interest. Lyophilization is a process by which the material to be dried is first frozen and then the ice or frozen solvent is removed by sublimation in a vacuum environment.

The cooling and drying process itself, however, can create stress that can cause protein degradation. Dimer and other insoluble protein aggregate indicate physical (non-covalent) aggregation is the likely cause of protein loss. So, a lyoprotectant must be included in pre-lyophilized formulations to enhance stability during the freeze-drying process and/or to improve stability of the lyophilized product upon storage.

When proteins are exposed to solvents, they may denature (by unfolding), forming irreversible aggregations. Denatured proteins tend to be more susceptible to irreversible chemical processes like proteolysis, oxidation and deamidation that will contribute to the loss of protein biological activities.

Consequently, it is an object of the present invention to provide formulations of bone morphogenetic proteins that can be lyophilized and are stable in organic solvents. The lyophilized formulation will be stable upon storage under normal storage condition. It is a further object to provide stable formulations of bone morphogenetic proteins that are stable in an organic solvent environment suitable for fabrication of a polymeric matrix for implantation and controlled local delivery of the protein.

### SUMMARY OF THE INVENTION

The invention is a protein formulation. The formulation comprises a bone morphogenetic protein, a lyoprotectant, and an amount of an oxidation/reduction stabilizer effective to stabilize the bone morphogenetic protein in the formulation.

Surprisingly, the addition of the oxidation/reduction stabilizer to the protein formulation significantly enhances the stability of the bone morphogenetic protein in the formulation relative to the stability which can be achieved without the oxidation/reduction stabilizer. The increased stability greatly increases the therapeutic effectiveness of the formulation.

In another embodiment, the invention is a medical device for delivering a protein. The device comprises a polymeric matrix and a protein formulation incorporated in the polymeric matrix. The protein formulation comprises a bone morphogenetic protein, a lyoprotectant, and an amount of oxidation/reduction stabilizer effective to stabilize the protein in the medical device.

In yet another embodiment, the invention is a coating for a medical device. The coating comprising a polymeric matrix and a protein formulation incorporated in the polymeric matrix. The protein formulation comprises a bone morphogenetic protein, a lyoprotectant, and an amount of an oxidation/reduction stabilizer effective to stabilize the protein in the coating.

The protein formulations, medical devices and coatings for medical devices of this invention can be used in all applications where bone growth induction is desired, particularly for orthopedic applications.

### DETAILED DESCRIPTION OF THE INVENTION

The bone morphogenetic proteins (BMPs) that may be used in the formulations of the present invention include, but are not limited to, TGF-β superfamily factors, BMP-2, BMP-4, BMP-6, BMP-7, BMP-12, BMP-14, and recombinant human growth differential factor 5 (rhGDF-5). The preferred BMP is rhGDF-5, which is also known as morphogenetic protein 52 (MP52). MP52 is manufactured by a process that yields product in the form of a solution. Solutions are commercially available in concentrations of up to 4.2 milligrams/milliliter.

Lyoprotectants enhance the stability of the BMP during the freeze-drying process. They also improve the stability of the lyophilized product upon storage. So, they must be included in pre-lyophilized formulations. Lyoprotectants that may be used in the formulations of the present invention include, but are not limited to, mannitol, sucrose, trehalose, and their blends. In a preferred embodiment, the lyoprotectant used in the present invention is mannitol. In the most preferred embodiment, the lyoprotectant is a blend of mannitol and sucrose. The weight ratio of bone morphogenetic proteins to lyoprotectants in the formulations of the present invention is between about 1:10 to about 1:40. Preferably, the ratio is between about 1:25 to about 1:35.

Oxidation/reduction stabilizers reduce the susceptibility of proteins to irreversible chemical processes like proteolysis, oxidation and deamidation that contribute to the loss of protein biological activities. This can occur during terminal sterilization of the formulation. We have demonstrated that these stabilizers enhance the stability of the protein. Oxidation/reduction stabilizers that may be used in the formulations of the present invention include, but are not limited to, Bovine Serum Albumin (BSA), Methionine, L-Arginine, Histidine, and cyclodextrin. The weight ratio of bone morphogenetic proteins to oxidation/reduction stabilizers in the formulations of the present invention is between about 1.2:1 to about 1:30. Preferably, the ratio is between about 1:3 to about 1:15.

Solvent environment stabilizers may be added to the formulations of the present invention. The stabilizers can help keep protein stable when exposed to organic solvents. Solvent environment stabilizers that may be used in the formulations of the present invention include, but are not limited to, polyoxyethylene sorbitan fatty acid esters. Preferably, the solvent environment stabilizer used in the present invention is a polyoxyethylene 20 sorbitan monooleate surface active agent sold under the tradename TWEEN 80 (ICI Americas Inc., Bridgewater N.J.). The weight ratio of bone morphogenetic proteins to solvent environment stabilizers in the formulations of the present invention is between about 7:1 to about 1:7. Preferably, the ratio is between about 1:1 to about 1:2.

Ideally, MP52 stock solution (usually in deep frozen form at −80° C.) is thawed slowly to about 4° C. The stock solutions of other ingredients with grades such as United States Pharmacopeias grade (USP) or European Pharmacopeias Grade (PhEur), are prepared in de-ionized water by simple mixing. MP52 stock solution, as well as the solutions of lyoprotectant and oxidation/reduction stabilizers are pipetted into a lyophilization vial. The solution is briefly vortexed to ensure homogeneity and placed into a freeze-dryer with an appropriate cooling and primary drying and secondary drying cycle. The ideal freeze-drying cycle is: primary drying at −40° C. for about 10 hours, followed by secondary drying at 15° C. and then 40° C. for a total of another eight hours. During drying, vacuum is maintained at 100 mTorr. After freeze-drying, a dry cake normally forms without any sign of shrinkage and collapse. The glass transition temperature of the white flaky cake is normally above 60° C. with a residual water content of less than 3% on a weight by weight basis. The preferred water content is about 1%.

Stable formulations of bone morphogenetic proteins that are amenable to organic solvents are suitable for fabrication of protein/polymer drug delivery devices for implantation and controlled local delivery of the protein. Such medical devices are characterized as having a polymeric matrix in which the protein formulations are incorporated. These devices include films, three-dimensional matrices, and microparticles. Stable formulations of protein can also be used as coatings on numerous types of implantable medical devices. In a similar fashion, these coatings have a polymeric matrix in which the protein formulation is incorporated.

In the case of films, the formulations of the present invention can be mixed into a polymer solution and the mixture can be cast into a film. When the solvent is removed, the polymeric film can be implanted locally to the site requiring the protein, and the protein can be released from the polymer matrix over weeks while remaining bioactive. In addition, the films may contain at least one pore forming agent, or porogen, which may be removed before, during or after implantation to yield a porous film to facilitate release of the protein.

To produce microparticles, the formulation can be encapsulated into a polymer particle via either conventional water/oil/water (w/o/w) double emulsion or through a spinning disk process that can atomize the polymer solution at the disk periphery by various physical means. Formulations also can be encapsulated into polymeric particles followed by grinding. Microparticles may also be made with porogens.

The formulations of the present invention can also be used as protein/polymer coatings on various implantable medical devices. Devices that can be coated with the protein/polymer coatings of this invention include, but are not limited to, sutures, needles, orthopedic pins, clamps, screws, plates, clips, staples, hooks, buttons, snaps, surgical instruments, vertebral discs, flowable grafts, and supports for cells in tissue engineering applications.

Polymers useful in the fabrication of the polymeric matrix for the medical devices and coatings for implantation and controlled local delivery of the protein may be either biodegradable or non-biodegradable. Biodegradable polymers readily break down into small segments when exposed to moist body tissue. The segments then either are absorbed by the body, or passed by the body. More particularly, the biodegraded segments do not elicit permanent chronic foreign body reaction because they are absorbed by the body or passed from the body, such that no permanent trace or residual of the segment is retained by the body.

Examples of biodegradable polymers and co-polymers that can be used in the protein/polymer drug delivery devices and coatings for implantation and controlled local delivery of the protein of the present invention include homopolymers, such as poly(glycolide), poly(lactide), poly(epsilon-caprolactone), poly(trimethylene carbonate) and poly(para-dioxanone); copolymers, such as poly(lactide-co-glycolide), poly(epsilon-caprolactone-co-glycolide), and poly(glycolide-co-trimethylene carbonate). The polymers may be statistically random copolymers, segmented copolymers, block copolymers or graft copolymers.

The following examples are illustrative of the principles and practice of this invention although not limited thereto. Numerous other embodiments within the scope and spirit of the invention will become apparent to those skilled in the art.

### EXAMPLE 1

### Formulations for MP52

MP52 stock solution (3.5 milligrams MP52 per milliliter of 0.01 Normal HCL aqueous solution), obtained from BIOPHARM GmbH (Heidelberg, Germany), was split into 200-microliter batches. Each 200-microliter batch of MP52 stock solution had 0.7 milligrams of MP52. To each batch, a combination of lyoprotectants, oxidation/reduction stabilizer, and solvent environment stabilizer were added in the amounts shown in Table 1 to create various formulations. The lyoprotectants were mannitol (EM Science, Darmstadt, Germany) and sucrose (Fisher, Fair Lawn, N.J.). The oxidation/reduction stabilizer was methionine (Sigma, St. Louis, Mo.). The solvent environment stabilizer was TWEEN 80 (Sigma, St. Louis, Mo.). The formulations were mixed and lyophilized through the following cycle: 1) primary drying step at −40° C., and 2) secondary drying step at 15° C. under a constant vacuum of 100 millitorr. The total freeze-dry time was around 20 hours. Typical water content in the dry formulation cake was 1% to 3% by weight.

The lyophilized samples were soaked in 200 microliters of methylene chloride (Mallinckrodt Baker Inc., Phillisburg, N.J.) and dried in a vacuum oven (23° C.) overnight. The dry samples were reconstituted in 0.01 Normal HCL aqueous solution and tested for protein quantity by reverse phase High Performance Liquid Chromatogaphy (RP-HPLC). The HPLC test was performed on an RP-HPLC system (Waters, Milford, Mass.) consisting of a pump (Model 510) and a UV detector (Waters 490E) with an auto sampler (Waters 717 Plus auto sampler). The HPLC system was equipped with a C18218TP54 Vydac Column (The Separation Group, Hesperia, Calif.). The column was eluted with mobile phase in gradient mode by mixing acetonitrile and 0.1% Trifluoroacetic acid at programmed ratio.

Table 1 shows the various formulations and the percent recovery of MP52 as determined by RP-HPLC.

TABLE 1
Formulations

| | | | | TWEEN | Methio- | Re- |
|---|---|---|---|---|---|---|
| | MP52 | Mannitol | Sucrose | 80 | nine | covery |
| Code | (mg) | (mg) | Mg | (mg) | (mg) | (%) |
| B-0 | 0.7 | 0 | 0 | 0 | 0 | 0 |
| B-1 | 0.7 | 1 | 0 | 1 | 0 | 0 |
| B-2 | 0.7 | 5 | 10 | 1 | 0 | 0 |
| B-3 | 0.7 | 5 | 20 | 1 | 0 | 0 |
| B-4 | 0.7 | 10 | 10 | 1 | 0 | 29 |
| B-5 | 0.7 | 10 | 20 | 1 | 0 | 44 |
| B-6 | 0.7 | 5 | 10 | 5 | 0 | 15 |
| B-8-8m-0 | 0.7 | 10 | 10 | 0 | 6 | 89 |
| B-8-8m-1 | 0.7 | 10 | 10 | 0.1 | 6 | 86 |
| B-8-8m-2 | 0.7 | 10 | 10 | 1 | 6 | 92 |

Table 1 shows that lyoprotectants (mannitol and sucrose) alone or in combination with solvent environment stabilizer (TWEEN 80) showed little (less than 50%) to no recovery of MP52 (Codes B-0 to B-6). When the oxidation/reduction stabilizer (methionine) was added to the lyoprotectants (alone or in combination with solvent environment stabilizer), recovery of MP52 increased to over 80%.

### EXAMPLE 2

### Comparison of Oxidation/Reduction Stabilizers

MP52 stock solution (3.5 milligrams MP52 per milliliter of 0.01 Normal HCL aqueous solution), obtained from BIOPHARM GmbH (Heidelberg, Germany), was split into 200-microliter batches. Each 200-microliter batch of MP52 stock solution had 0.7 milligrams of MP52. To each batch, a combination of lyoprotectants, oxidation/reduction stabilizer, and solvent environment stabilizer were added in the amounts shown in Tables 2-6 to create various formulations.

The lyoprotectants were mannitol (EM Science, Darmstadt, Germany) and sucrose (Fisher, Fair Lawn, N.J.). The oxidation/reduction stabilizers were L-histidine, L-arginine, cyclodextrin, Bovine Serum Albumin (BSA), and methionine (all from Sigma, St. Louis, Mo.). The solvent environment stabilizer was TWEEN 80 (Sigma, St. Louis, Mo.).

The formulations were mixed and lyophilized as described in Example 1. Typical water content in the dry formulation cake was 1% to 3%. As described in Example 1, the lyophilized samples were soaked in 200 microliters of methylene chloride and dried in a vacuum oven (23° C.) overnight. The dry samples were reconstituted in 0.01 Normal HCL aqueous solution and tested for protein quantity by reverse phase High Performance Liquid Chromatogaphy (RP-HPLC).

Tables 2 through 6 show the various formulations and the percent recovery of MP52 as determined by RP-HPLC.

TABLE 2
L-histidine Formulations

| | | | | | | Re- |
|---|---|---|---|---|---|---|
| | MP52 | Mannitol | Sucrose | TWEEN | l-histidine | covery |
| Code | (mg) | (mg) | (mg) | 80 (mg) | (mg) | (%) |
| B-8-1h | 0.7 | 10 | 10 | 5 | 1 | 36 |
| B-8-2h | 0.7 | 10 | 10 | 5 | 2 | 37 |
| B-8-3h | 0.7 | 10 | 10 | 5 | 5 | 41 |
| B-8-4h | 0.7 | 10 | 10 | 5 | 10 | 51 |

TABLE 3
L-arginine Formulations

| | | | | | l- | |
|---|---|---|---|---|---|---|
| | MP52 | Mannitol | Sucrose | TWEEN | arginine | Recovery |
| Code | (mg) | (mg) | (mg) | 80 (mg) | (mg) | (%) |
| B-8-5a | 0.7 | 10 | 20 | 5 | 0.5 | 40 |
| B-8-6a | 0.7 | 10 | 10 | 5 | 1 | 39 |
| B-8-7a | 0.7 | 10 | 10 | 5 | 3 | 38 |
| B-8-8a | 0.7 | 10 | 10 | 5 | 6 | 61 |
| B-8-9a | 0.7 | 10 | 10 | 5 | 10 | 60 |

TABLE 4
cyclodextrin Formulations

| | | | Su- | | | Re- |
|---|---|---|---|---|---|---|
| | MP52 | Mannitol | crose | TWEEN | cyclodextrin | covery |
| Code | (mg) | (mg) | (mg) | 80 (mg) | (mg) | (%) |
| B-8-1c | 0.7 | 10 | 10 | 5 | 1 | 38 |
| B-8-2c | 0.7 | 10 | 10 | 5 | 2 | 37 |
| B-8-3c | 0.7 | 10 | 10 | 5 | 5 | 38 |
| B-8-4c | 0.7 | 10 | 10 | 5 | 10 | 62 |

TABLE 5
Bovine Serum Albumin (BSA)Formulations

| | | Mannitol | Sucrose | TWEEN | BSA | Recovery |
|---|---|---|---|---|---|---|
| Code | MP52 (mg) | (mg) | (mg) | 80 (mg) | (mg) | (%) |
| B-6 | 0.7 | 5 | 10 | 5 | 0 | 15 |
| B-7 | 0.7 | 5 | 20 | 5 | 1 | 30 |
| B-8 | 0.7 | 10 | 10 | 5 | 10 | 102 |
| B-9 | 0.7 | 10 | 20 | 5 | 0 | 14 |

TABLE 6
Methionine Formulations

| | | | | | Methio- | |
|---|---|---|---|---|---|---|
| | MP52 | Mannitol | Sucrose | TWEEN | nine | Recovery |
| Code | (mg) | (mg) | (mg) | 80 (mg) | (mg) | (%) |
| B-8-5m | 0.7 | 10 | 20 | 5 | 0.5 | 78 |
| B-8-6m | 0.7 | 10 | 10 | 5 | 1 | 75 |
| B-8-7m | 0.7 | 10 | 10 | 5 | 3 | 77 |
| B-8-8m | 0.7 | 10 | 10 | 5 | 6 | 77 |
| B-8-9m | 0.7 | 10 | 10 | 5 | 10 | 81 |

The tables show that all of the oxidation/reduction stabilizers tested showed 5 the ability to enhance recovery of MP52. The Oxidation/reduction stabilizers methionine and BSA showed the greatest ability to enhance recovery of MP52.

### EXAMPLE 3

### Methionine as an Oxidation/Reduction Stabilizer

Formulations containing MP52, lyoprotectants (mannitol and sucrose), oxidation/reduction stabilizer (methionine), and solvent environment stabilizer (TWEEN 80) were mixed and lyophilized as discussed in Examples 1 and 2. Typical water content in the dry formulation cake was 1% to 3%. As described in Examples 1 and 2, the lyophilized samples were soaked in 200 microliters of methylene chloride and dried in a vacuum oven (23° C.) overnight, then reconstituted in 0.01 Normal HCL aqueous solution and tested for protein quantity by RP-HPLC. Table 7 shows the formulations, as well as the percent recovery of MP52 as determined by RP-HPLC.

TABLE 7
Methionine Formulations

| | | | | Methio- | TWEEN | Re- |
|---|---|---|---|---|---|---|
| | MP52 | Mannitol | Sucrose | nine | 80 | covery |
| Code | (mg) | (mg) | Mg | (mg) | (mg) | (%) |
| B-8-8m-0 | 0.7 | 10 | 10 | 6 | 0 | 89 |
| B-8-8m-1 | 0.7 | 10 | 10 | 6 | 0.1 | 86 |
| B-8-8m-2 | 0.7 | 10 | 10 | 6 | 1 | 92 |
| B-8-8m-3 | 0.7 | 10 | 10 | 6 | 6 | 59 |

Table 7 shows that a combination of methionine and small amount of TWEEN 80 are effective in providing high MP52 protein recovery. The table also shows that a higher level of TWEEN 80 is not beneficial.

### EXAMPLE 4

### MP52 Release from Polymer Films

Poly(lactide-co-glycolide), or 50/50 PLGA polymer (Alkermes, Cincinnati, Ohio), with an intrinsic viscosity (I.V.) of 0.25-0.43 dl/gram, was mixed with methylene chloride at 30° C. to create a 10 weight percent polymer solution. A pore forming agent, poloxomer 188 (BASF, Mt. Olive, N.J.) was then added to the polymer solution at a quantity of 177.5 milligrams for every 5 grams of polymer solution. Poloxomer, also called Pluronic F68, is a block copolymer of ethylene oxide and propylene oxide.

Lyophilized formulation cake B-8-8m-2 (see Example 3, Table 7) was triturated into small particles. The reduced cake was mixed with the polymer solution by mild vortex at two levels, 17 weight percent (high) and 5.25 weight percent (low), to form uniform suspensions. The solutions were cast into a TEFLON-coated molds and air dried in a laminar flow hood at room temperature.

The dried films were tested for MP52 release in a release media of 20 millimolar sodium acetate with 0.1 percent Bovine Serum Albumin (pH 4.0). The release media was replenished at each time point, with the old release media being tested for MP52 by RP-HPLC as described in Example 1.

TABLE 8
MP52 Cumulative Release from polymer film

| Time (hrs) | Low | High |
|---|---|---|
| 0 | 0.0 | 0% |
| 2.5 | 26% | 63% |
| 6 | 38% | 73% |
| 24 | 50% | 77% |
| 48 | 61% | 80% |

Table 8 shows the percent cumulative release of MP52 from protein-loaded films. The table shows, under accelerated release condition, the MP52 protein can be recovered intact. The percentage release has positive correlation with the protein loading, with higher loaded film releasing protein about 80% during the 48 hours accelerated study and the lower loaded film releasing only 61% during the same time interval.

### EXAMPLE 5

### MP52-Containing Microspheres

Twelve units of formulations were prepared, with each unit of formulation containing 2.8 mg of MP52, 10 mg each of mannitol and sucrose (lyoprotectants), 6 mg of methionine (oxidation/reduction stabilizer), and 1 mg of TWEEN 80 (solvent environment stabilizer). The formulations were each mixed and lyophilized as discussed in Examples 1 and 2. Typical water content in the dry formulation cake was 1% to 3%. The lyophilized formulation cake was triturated into small particles.

In addition, 8 grams of 50/50 PLGA polymer (Alkermes, Cincinnati, Ohio) with an intrinsic viscosity (I.V.) of 0.66 to 0.80 dl/gm was dissolved in methylene chloride to make a 5 percent (w/w) polymer solution.

The twelve units of formulation were suspended in the polymer solution by votexing at high speed for one minute.

The Spinning disk set-up was as follows: the temperature at the top of the chamber was set at 58 to 60° C.; the temperature at the bottom of the chamber, where the particles were collected, was set at 45 to 47° C. The diameter of the disk was 3 inches. Polymer solution suspended with protein powder was fed at a rate of 150 grams/minute with disk spinning at a speed of 4,000 rpm. After microsphere atomization from the system, microspheres were collected with a glass jar on a cyclone in the blow out disk chamber under nitrogen.

The microspheres were screened, and those under 65 micron in size were collected. To recover the MP52, the microspheres were placed in methylene chloride, which dissolved away the polymer phase and precipitated out the MP52. The MP52 was then dissolved in a 6 M urea and phosphate buffer system (pH=2.75). Reverse phase High Performance Liquid Chromatogaphy (RP-HPLC) analysis was used to test for protein quantity as described above. RP-HPLC analysis determined that up to about 80% of the initial MP52 loaded into the microspheres was recovered.

What is claimed is:

1. A protein formulation comprising a bone morphogenetic protein, a lyoprotectant, and an amount of an oxidation/reduction stabilizer effective to stabilize the bone morphogenetic protein in the formulation.

2. The protein formulation of claim 1 wherein the bone morphogenetic protein is selected from the group consisting of TGF-β superfamily factors, BMP-2, BMP-4, BMP-6, BMP-7, BMP-12, BMP-14, and rhGDF-5.

3. The protein formulation of claim 2 wherein the bone morphogenetic protein is rhGDF-5.

4. The protein formulation of claim 1 wherein the lyoprotectant is selected from the group consisting of mannitol, sucrose, and trehalose.

5. The protein formulation of claim 4 wherein the lyoprotectant is a blend of mannitol and sucrose.

6. The protein formulation of claim 1 wherein the oxidation/reduction stabilizer is selected from the group consisting of bovine serum albumin, methionine, L-arginine, histidine, and cyclodextrin.

7. The protein formulation of claim 6 wherein the oxidation/reduction stabilizer is methionine.

8. The protein formulation of claim 1 wherein the formulation further comprises a solvent environment stabilizer.

9. The protein formulation of claim 8 wherein the solvent environment stabilizer is selected from the group consisting of polyoxyethylene sorbitan fatty acid ester and polyoxyethylene 20 sorbitan monooleate.

10. The protein formulation of claim 1 wherein the amount of the oxidation/reduction stabilizer in the formulation is defined by a weight ratio of bone morphogenetic protein to oxidation/reduction stabilizer between about 1.2:1 to about 1:30.

11. The protein formulation of claim 10 wherein the weight ratio of bone morphogenetic protein to oxidation/reduction stabilizer is between about 1.3 to about 1:15.

12. A medical device for delivering a protein, the device comprising a polymeric matrix and a protein formulation incorporated in the polymeric matrix, wherein the protein formulation comprises a bone morphogenetic protein, a lyoprotectant, and an amount of oxidation/reduction stabilizer effective to stabilize the protein in the medical device.

13. The medical device of claim 12 wherein the protein formulation further comprises a solvent environment stabilizer.

14. The medical device of claim 12 wherein the polymeric matrix is composed of a biodegradable polymer.

15. The medical device of claim 14 wherein the biodegradable polymer is selected from the group consisting of poly(glycolide), poly(lactide), poly(epsilon-caprolactone), poly(trimethylene carbonate), poly(para-dioxanone), poly(lactide-co-glycolide), poly(epsilon-caprolactone-co-glycolide), and poly(glycolide-co-trimethylene carbonate).

16. The medical device of claim 12 wherein the device is in the form of a film, a three-dimensional matrix or a microparticle.

17. A coating for a medical device, the coating comprising a polymeric matrix and a protein formulation incorporated in the polymeric matrix, wherein the protein formulation comprises a bone morphogenetic protein, a lyoprotectant, and an amount of an oxidation/reduction stabilizer effective to stabilize the protein in the coating.

18. The coating of claim 17 wherein the formulation further comprises a solvent environment stabilizer.

19. The coating of claim 18 wherein the medical device is selected from the group consisting of sutures, needles, orthopedic pins, clamps, screws, plates, clips, staples, hooks, buttons, snaps, surgical instruments, vertebral discs, flowable grafts and supports for cells in tissue engineering applications
